(19) United States
(12) Patent Application Publication
(10) Pub. No.: US 2024/0091503 A1
(43) Pub. Date: Mar. 21, 2024

(54) CATHETER STABILIZER AND GRIP

(71) Applicant: Walter Kusumoto, Chico, CA (US)

(72) Inventor: Walter Kusumoto, Chico, CA (US)

(21) Appl. No.: 18/370,999

(22) Filed: Sep. 21, 2023

Related U.S. Application Data
(60) Provisional application No. 63/408,692, filed on Sep. 21, 2022.

(51) Int. Cl. A61M 25/02

(52) U.S. Cl. CPC A61M 25/02; A61M 2025/024; A61M 2025/0253; A61M 2025/026; A61M 2025/0266

(57) ABSTRACT

A grip assembly and pad are removably attachable to each other for immobilization of a catheter attached to the grip assembly. Removable attachability is provided between the catheter and pad through interaction of complementary portions of a hook and loop, fastener, and optionally utilizing an additional mechanical fastener associated with the pad. In some embodiments, the mechanical fastener can be the only catheter engagement, and the catheter can either be engaged, directly or through the grip assembly. The pad can be replaced with other catheter anchors which firmly grip the catheter and hold it against rotation, and/or translation. The catheter is thus readily and easily transitioned between a stabilized state where translation and rotation are resisted, and a freely movable state where the catheter can be readily rotated and translated by a surgeon, or other medical professional.

### CROSS-REFERENCE TO RELATED APPLICATIONS

This application claims benefit under Title 35, United States Code § 119(e) of U.S. Provisional Application No. 63/408,692 filed on Sep. 21, 2022.

### FIELD OF THE INVENTION

The following invention relates to accessories attachable to catheters, such as cardiac catheters, to allow the catheters to be readily moved, positioned and oriented as desired, and then to maintain the state in which the catheter has been placed. More particularly, this invention relates to a catheter stabilizer which allows the catheter to be held immobile by temporary fastening to an anchor pad or other motion resisting reference structure, and to be readily disengaged when desired for further catheter intentional repositioning.

### BACKGROUND OF THE INVENTION

Flexible catheters have been used in medicine since the 18^{th} century, and modern disposable catheters have been used since the 1940s. In modern day medicine, catheters are used in the cardiac electrophysiology lab, cardiac catheterization suite, interventional radiology suite, operating room and other hospital locations for diagnostic and treatment purposes. Modern catheters are shaped, have curvatures and composed of materials so that the distal tip can be placed in specific anatomic locations with minimal effort to achieve their diagnostic and therapeutic functions. However, with some frequency, the operator/proceduralist needs to maintain torque/tension or push force on the catheter in order to maintain positioning and orientation of the distal tip of the catheter so that they may perform their diagnostic and therapeutic goals. This requires one of the hands of the operator or assistant to maintain torque on the catheter so that the catheter may stay in position within the body. Sometimes an instrument such as a hemostat or tape is used to maintain torque on the catheter, however this frequently causes catheter damage, and many times does not maintain adequate rotational/tension or push force on the catheter for the duration of the procedure or the hospital stay. Furthermore, and with the use of a hemostat, this hemostat can itself still move, somewhat along with the catheter, unless the hemostat itself is coupled to some anchor, or has sufficient mass to resist such a motion. Thus, the use of a hemostat to resist rotation, and/or translation of the hemostat and catheter it's not entirely effective when used during a medical procedure. Also, after the medical procedure is complete, and it is desired to keep the catheter in a fixed state, and without rotation or translation relative to the body of the patient, the hemostat is a poor choice to remain with the patient in a post procedure long term manner.

It is known to place sutures between the catheter and skin of the patient to keep the catheter without motion, especially after the medical procedure involving the catheter has been completed. This suturing procedure takes time for the medical professional to properly complete. Furthermore, catheters are not generally well configured to be coupled to sutures. Also, catheters have a generally elongate cylindrical form, and a generally smooth outer surface, which causes them to have a high propensity towards rotation about their long axis, even if sutures are placed in an attempt to hold the catheter against such rotation or translation.

In the case of the option of using tape to resist translation and/or rotation, adhesive forces are often insufficient to resist the translation and rotation forces impart upon the catheter by the particular positioning of a distal tip of the catheter within a body lumen of the patient. If the tape is strong enough to resist such translation and rotation forces (and torques) initially, adhesive on tape has a tendency to diminish in holding strength over time, as skin to which the tape is attached naturally sheds its outer layers, sweats and/or is flexed. Adhesive can also lose holding strength merely due to passage of time, during catheter entry point cleaning and maintenance, and due to other deteriorating influences.

Accordingly, a need exists for a catheter grip/tool, which is easily engaged and disengaged from the catheter. Such a grip/tool would be readily attachable and detachable to an anchor structure, such as an anchor pad, which can then be attached to skin, or to surgical drape, or to other low mobility (e.g. large) structures. Such a tool would be useful during performance of medical procedures to hold the catheter precisely were desired, when lack of translation and/or rotation is desired. Then, after the procedure is complete, such fixation of the catheter can be maintained post procedure, and without requiring complex equipment and/or further procedures, such as suturing (or to at least simplify such further suturing/fixation procedures).

### SUMMARY OF THE INVENTION

With this invention, a system is provided for stabilizing a catheter during and after use. The system is convenient to attach to a catheter, and simple and convenient to engage and disengage between a stabilized state and a freely movable state. In the stabilized state, the system holds the catheter to resist translation and/or rotation of the catheter relative to the body, holding the position that it was in when the system was engaged. In the freely movable state, a medical professional can translate and rotate the catheter according to the judgment of the medical professional. The system allows for exceptionally rapid transition between the stabilized state, and the freely movable state, to optimize convenience in support of the medical procedure being performed by the surgeon or other medical professional.

The system includes an anchor pad or other anchor element as well as a grip which is attachable to or incorporated into the catheter. The grip and the anchor element are configured so that they can be readily attached and detached relative to each other. A fastener is provided on the anchor element, the grip or both, which fastener can be engaged and disengaged to transition the system from the stabilized state to the freely movable state, and vice versa, in a repeatable fashion. In one embodiment, the fastener includes complementary portions of a hook and loop type fastener, such as that provided under the trademark VELCRO, by the Velcro IP Holdings LLC of Manchester, New Hampshire.

The anchor, when in the form of an anchor pad, can be provided as a form of surgical drape, or as a flexible layer which can be attachable to surgical drape, such as through an adhesive backing on a back surface of the anchor pad. As another option, this back surface can attach to skin of a patient through an adhesive thereon. The anchor can optionally resist translation and/or rotation, primarily by being sufficiently heavy and sufficiently large and flat in size and shape, that it can resist rotation merely by laying upon an underlying surface and in an at least somewhat flattened orientation, and resist the torques and linear forces which might be exerted thereon by the catheter. In this way, catheter translational position and rotational orientation can be maintained where last placed by the surgeon or other medical professional. In at least some embodiments, an anchor pad can be provided which also includes a mechanical fastener thereon, either in place of the hook and loop fastener, or in addition to the hook and loop fastener. Examples of such mechanical fasteners placed upon the anchor pad can include belts, clamps, clips, clasps, sleeves, gates and other mechanical fasteners. Anchors which do not strictly require a pad can include cam clamps, vee clamps and scissor clamps.

The catheter can have a grip assembly removably mounted thereto, which is configured to be attachable to the anchor pad, or other anchor. For instance, and in one embodiment, the grip assembly can have an outer surface with a complementary portion of the hook and loop fastener of the pad located thereon. With such a configuration, the grip assembly, when anchored to the catheter, can merely be pressed down somewhat onto the anchor pad, and it will maintain its position, both rotationally and translationally. This can occur merely by setting down the catheter and associated grip assembly, or optionally pressing downward slightly to enhance engagement of the hook and loop fastener.

The grip assembly can have a variety of different configurations for removable attachment to the catheter. Optionally, this grip assembly could be integrated into the catheter itself or made, at least semi permanently attachable to the catheter. In other embodiments, the anchor or mechanical fasteners associated with the anchor pad can be configured to grip and hold an unmodified catheter thereto, so that the outer surface of the catheter acts as a form of grip in such embodiments. Different trade-offs and preferences can be accommodated by selecting an appropriate anchor pad, with or without a mechanical fastener associated therewith, or an anchor and a corresponding grip, assembly, or other grip, either attachable to the catheter, integrated into the catheter, or working with an exterior surface of an existing catheter.

One example embodiment of grip assembly includes a body and a cap which both have portions of a central bore passing therethrough. The body and cap are threaded onto the catheter from one of the ends of the catheter, typically before a medical procedure involving the catheter is commenced. An appropriate location on the catheter, which is expected to remain outside of the body of the patient but near a trans cutaneous access site, is selected for attachment of the grip assembly to the catheter. The body and cap in one embodiment are configured with complementary male and female threads thereon to thread together.

A catheter engagement is activated by tightening of the cap onto the body. This catheter engagement can be a series of fingers extending from an end of the base closest to the cap, which are compressed down against the catheter by the cap, when the cap is threaded onto the base. Another catheter engagement could be in the form of a compressible washer, which also girds the catheter, and is located between the cap and the base. When the cap is threaded onto the threads of the base, the washer is compressed and caused to have a hole therein to shrink in size, and to firmly grip the catheter.

An exterior of the grip assembly is provided with loops (or hooks) of a hook and loop fastener system. Complementary hooks (or loops) are provided upon a top surface of an anchor pad, which has its back surface adhesively or otherwise attached to surgical drape or to skin of a patient near the transcutaneous access site for the catheter. During use, and when the catheter has been placed translationally and rotationally precisely were desired, a surgeon, can merely press the grip assembly (which is mounted to the catheter) down onto the anchor pad, and the hook and loop fastener engages to hold the catheter in its stabilized state, without appreciable translation and/or rotation. If movement of the catheter is desired, the catheter can be easily lifted to cause the grip assembly and its associated hook and loop fastener portion to disengage from the anchor pad, and the catheter can be rotated and/or translated as needed. It can then be readily pressed back down onto the anchor pad to again return the catheter to its stabilized state.

If the catheter needs long-term maintenance in its stabilized state, suture loops can be utilized to place a suture between the grip assembly, and either patient skin or surgical drape or other less structures. As an alternative, the anchor pad can be provided with a mechanical fastener in addition to the hook and loop fastener, which can be engaged to most securely hold the catheter precisely where desired for long-term catheter stabilization, and resistance to various tugging forces, or potential for tampering, such as by a delirious patient.

### OBJECTS OF THE INVENTION

Accordingly, primary object of the present invention is to provide a system for immobilizing a catheter from rotation and/or translation, while a portion of the catheter extends transcutaneously into a body lumen of a patient.

Another object of the present invention is to provide a system, including an anchor portion and a catheter gripping portion, with a fastener therebetween for removably and securely holding the catheter relative to the anchor.

Another object of the present invention is to provide a method for easily and quickly immobilizing a catheter in a desired position and orientation.

Another object of the present invention is to provide a grip assembly which can be readily attached to a catheter, and which grip assembly can be removably attached to an anchor, such as an anchor pad.

Another object of the present invention is to provide a grip assembly which can be removably attached to an anchor pad, which is itself attached in a fixed position relative to a patient, such as through attachment to patient skin or to surgical drape upon the patient.

Another object of the present invention is to provide an anchor which can hold a catheter in a desired position and orientation, by the anchor having sufficient mass and shape to resist rotation when the anchor is resting upon a surface.

Another object of the present invention is to provide a catheter stabilizer which can receive sutures for further holding the catheter to skin, or to other structures for immobilization of the catheter.

Another object of the present invention is to place a catheter in a stabilized state, and to quickly and easily transition the catheter into a freely movable state, and quickly and easily transition the catheter back into and stabilized state, repeatedly.

Another object of the present invention is to provide a catheter stabilization system which is removably attachable to catheters of different types and styles, as well as catheter accessories such as sheaths.

Another object of the present invention is to provide a catheter stabilization system which can work with an unmodified catheter or with a permanently or temporarily modified catheter.

Other further objects of the present invention will become apparent from a careful reading of the included drawing figures, the claims and detailed description of the invention.

### BRIEF DESCRIPTION OF THE DRAWINGS

FIG. 1 is a perspective, view of a pad and a front elevation view of a grip assembly and showing how a catheter can be inserted into the grip assembly and the grip assembly, once attached to the catheter, can be readily attached to the pad, and which pad can be readily attached to an underlying reference surface.

FIG. 2 is a hybrid view similar to that which is shown in FIG. 1, but after the grip assembly has been attached to the catheter.

FIG. 3 is a perspective view of an anchor pad and grip assembly, according to an embodiment where a slit allows for opening of at least a body of the grip assembly to receive the catheter laterally thereinto.

FIG. 4 is a front elevation exploded view of the grip assembly of FIGS. 1 and 2, and featuring fingers as a catheter engagement.

FIG. 5 is a front elevation view similar to that which is shown in FIG. 4, but after closure of a cap of the grip assembly onto a body of the grip assembly, for securing the grip assembly to the catheter.

FIG. 6 is a front elevation exploded view of the grip assembly of FIGS. 1 and 2, in an embodiment featuring a compressible washer for securing the grip assembly to the catheter.

FIG. 7 is a front elevation view similar to that which is shown in FIG. 6, but after closure of a cap of the grip assembly onto a body of the grip assembly for securing of the grip assembly to the catheter.

FIG. 8 is a perspective view similar to that which is shown in FIG. 2, but with the pad further including a belt thereon as one form of mechanical fastener, provided in addition to (or as an alternative to) a hook and loop fastener.

FIG. 9 is a perspective view similar to that which is shown in FIG. 8, but featuring a clamp as an alternative form of mechanical fastener.

FIG. 10 is a perspective view similar to that which is shown in FIG. 8, but featuring a clip as an alternative form of mechanical fastener.

FIG. 11 is a perspective view similar to that which is shown in FIG. 8, but featuring a clasp as an alternative form of mechanical fastener.

FIG. 12 is a perspective view similar to that which is shown in FIG. 8, but for an unmodified catheter, and with a mechanical fastener in the form of a sleeve into which the catheter can be wedged for securely holding the catheter without rotation and/or translation.

FIG. 13 is a perspective views similar to that was shown in FIG. 12, but featuring a clasp instead of a sleeve.

FIG. 14 is a perspective view similar to that which is shown in FIG. 12, but featuring a belt instead of a sleeve.

FIG. 15 is a perspective view similar to that which is shown in FIG. 12, but featuring a clamp instead of a sleeve.

FIG. 16 is a perspective view similar to that which is shown in FIG. 12, but featuring a clip instead of a sleeve.

FIG. 17 is a perspective view similar to that which is shown in FIG. 12, but featuring a gate as a mechanical fastener instead of a sleeve.

FIG. 18 is an end elevation view of a locking clip, providing an alternative embodiment mechanical fastener instead of the sleeve of FIG. 12.

FIG. 19 is a perspective view of that which is shown in FIG. 18.

FIG. 20 is a top plan view of a cam clamp and catheter exploded from the cam clamp, and illustrating how the catheter can be placed into the cam clamp for immobilization thereof, the cam clamp having sufficient size and mass to function as an anchor and cause the catheter to resist rotation and/or translation when the cam clamp is resting upon an underlying service.

FIG. 21 is a top plan view similar to that which is shown in FIG. 20, but after the catheter has been engaged with the cam clamp.

FIG. 22 is an end elevation view of a vee clamp configured to releasably hold a catheter, the vee clamp shown in a closed configuration and having sufficient mass and shape to act as an anchor and resist rotation for itself and an attached catheter, when resting upon an underlying service.

FIG. 23 is an end elevation view of that which is shown in FIG. 22, but with the vee clamp shown in an open configuration.

FIG. 24 is an end elevation view of a scissor clamp configured to releasably hold a catheter, the scissor clamp shown in a closed configuration and having sufficient mass and shape to act as an anchor and resist rotation for itself and an attached catheter, when resting upon an underlying surface.

FIG. 25 is an end elevation view of that which is shown in FIG. 24, but with the scissor clamp shown in an open configuration.

FIG. 26 is a perspective view of a catheter which includes a grip surface, or at least a portion thereof, so that such a specialty catheter can attach directly to an underlying anchor pad, such as through a hook and loop fastener, including a portion on the anchor pad and a portion on the grip surface of the catheter.

FIG. 27 is a perspective view of an anchor pad and a catheter, with the catheter including a grip sheet in the process of being attached to an outer surface of the catheter, and in broken lines shown after being attached to an outer surface of the catheter, the grip sheet configured to attach removably to the anchor pad, such as through enmeshing of complementary hook and loop elements.

FIG. 28 is a perspective view of an anchor pad and a catheter, with a grip clip formed of resilient material and having a hollow interior and a slit, so that the grip clip is able to snap onto the catheter, and also showing the grip clip outline after it has been attached to the catheter.

### DESCRIPTION OF THE PREFERRED EMBODIMENT

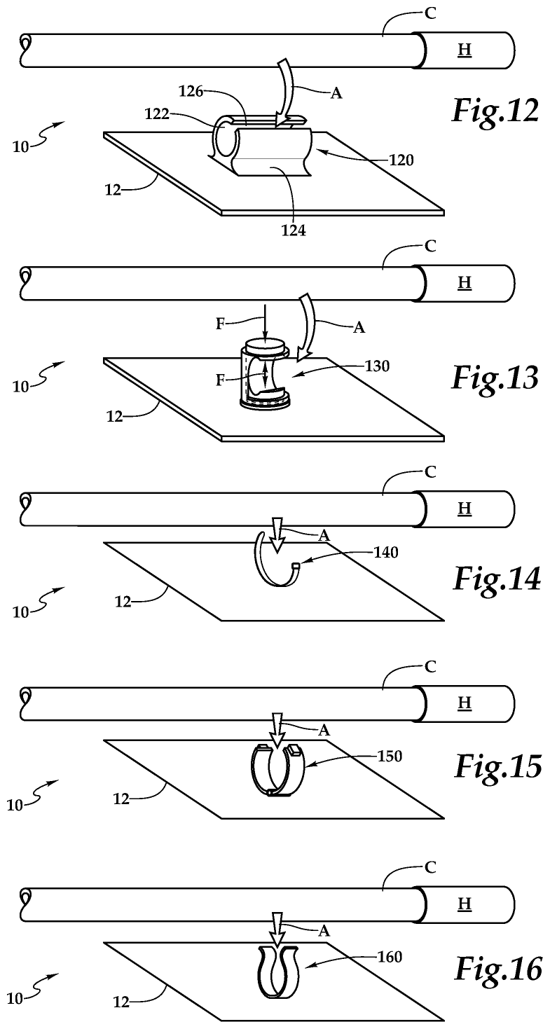

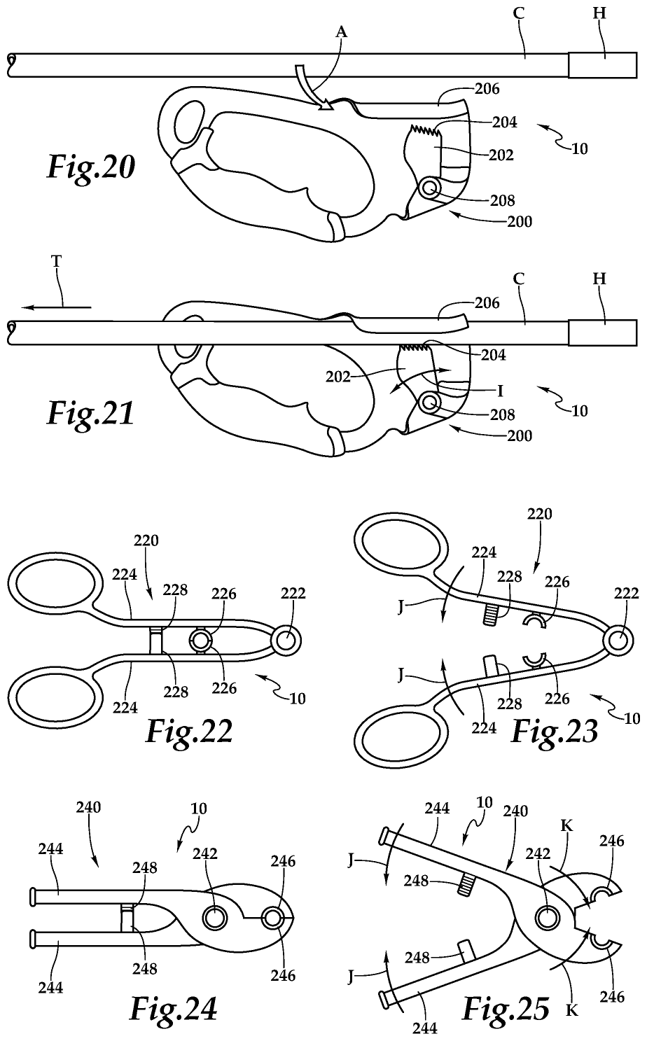

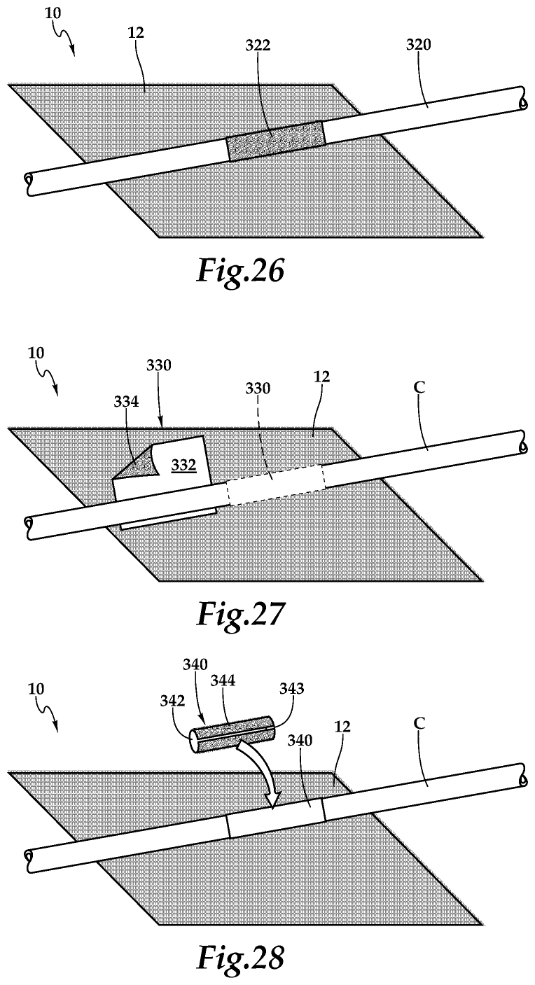

Referring to the drawings, wherein like reference numerals represent like parts throughout the various drawing figures, reference numeral 10 is directed to a system for stabilizing a catheter C. The system 10 (FIGS. 1-11) utilizes a grip assembly 20 in many embodiments and variations, which is removably attachable to the catheter C and is configured to be attachable to an anchor pad 12 or other anchor (FIGS. 20-25), for immobilization of the catheter C against rotation and/or translation. In certain embodiments (FIGS. 12-19, 27 and 28), an unmodified catheter C (or the modified catheter 320 of FIG. 26) can be removably attached to an anchor, or mechanical fastener of an anchor pad 12, or directly to the anchor pad 12 for similar immobilization of the catheter against rotation and/or translation.

In essence, and with particular reference to FIGS. 1-7, basic details of the system 10 are disclosed, according to an example embodiment and variations thereof. The system 10 includes the anchor pad 12, which can attach to a reference surface 15, such as skin or surgical drape. The grip assembly 20 is removably attachable to the catheter C. A fastener, such as hook and loop fastener, is provided on the pad 12 and grip assembly 20 to allow for repeated easy and fast attachment and detachment of the grip assembly 20 to the pad 12, for transitioning the catheter C between a stabilized state and a freely moveable state. The grip assembly 20 includes a body 30 and a cap 40 in this embodiment, which thread together. A catheter engagement, such as resilient fingers 50 or a compressible washer 60, are interposed between the body 30 and the cap 40 to cause the grip assembly 20 to be removable attachable to the catheter C.

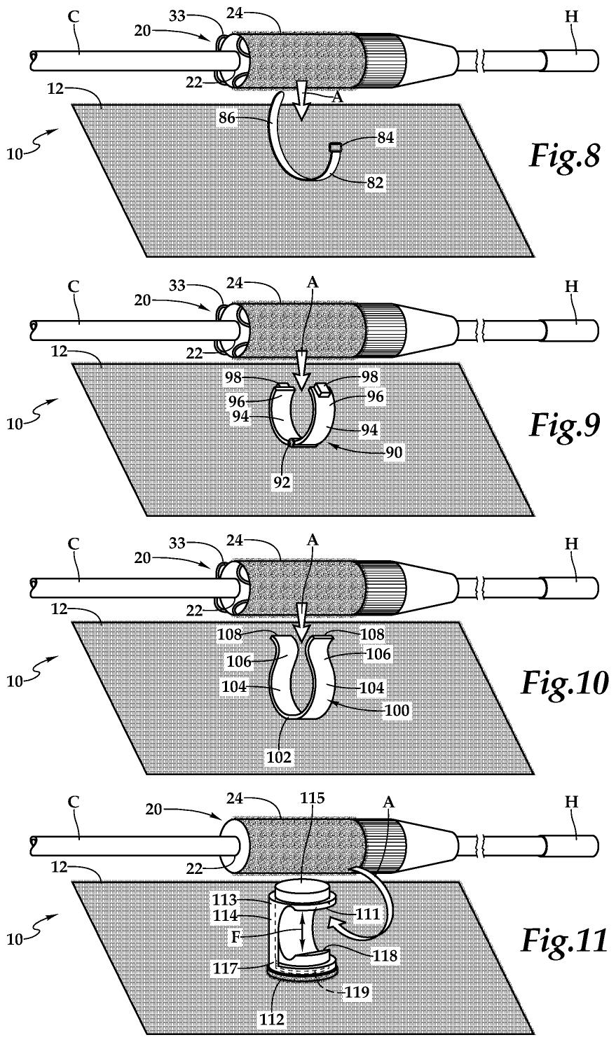

The pad 12 can be configured to additionally include a mechanical fastener (FIGS. 8-11) to more securely hold the grip assembly 20. This mechanical fastener can be a belt 80, clamp 90, clip 100, clasp 110, sleeve 120, gate 170, locking clip 180 or other mechanical fastener. Such mechanical fasteners can be provided on the pad 12 without the hook and look fastener as well (FIGS. 12-19), and can act upon the catheter C without the grip assembly 20 (FIGS. 12-25). The pad 12 can be replaced with an anchor, such as a cam clamp 200, vee clamp 220 or scissor clamp 240. A modified catheter 320 can have an integrated grip surface 322 to function as a grip assembly 20 replacement. Alternatively, the catheter C can be modified to include a grip sheet 330 or a grip clip 340 thereon to facilitate coupling to the pad 12 directly (or nearly directly), such as with complementary hook and loop fasteners.

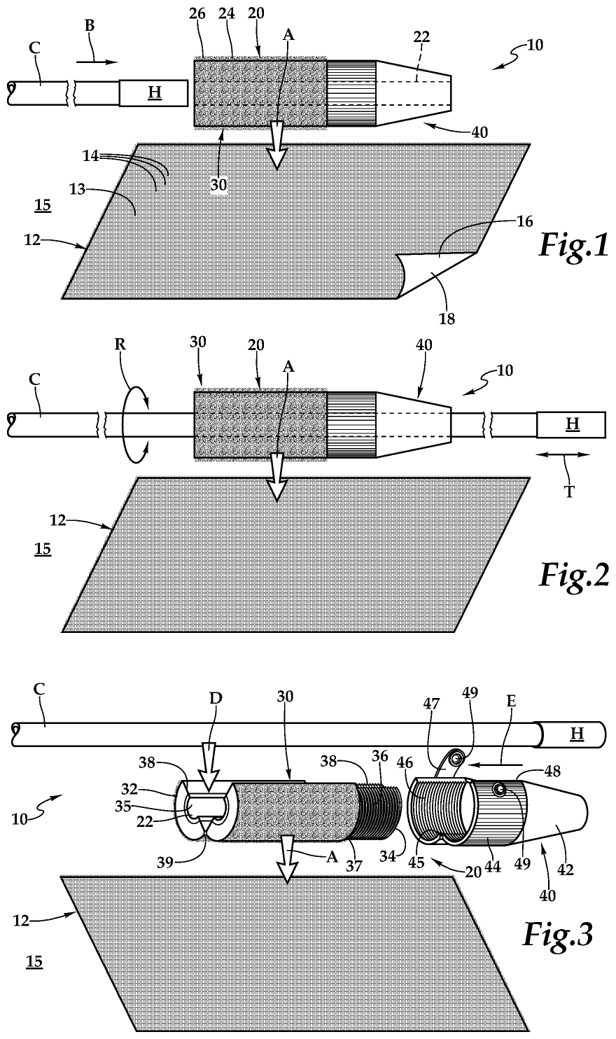

More specifically, and with continuing reference to FIGS. 1 and 2, basic details of the system 10, including the pad 12 and grip assembly 20, are described according to an example embodiment and variations thereof. The pad 12 (also referred to as an anchor pad) acts as one portion of the system 10 of this invention. This pad 12 can operate alone or with various different mechanical fasteners (FIGS. 8-11) to assist in holding the grip assembly 20. The mechanical fasteners can be modified to hold directly to the catheter C in other embodiments and variations (FIGS. 12-19). As an alternative to the pad 12, other anchors can be utilized (FIGS. 22-25). A catheter 320 can be modified to have some attributes of the grip assembly 20 built into the catheter 320, so that the catheter 320 can interact directly with the pad 12 (FIG. 26). Alternatively, the catheter C can be modified slightly to have attributes similar to those of the grip assembly 20, for use with the pad 12.

The pad 12 includes a top surface 13 opposite of back surface 16. The pad 12 is a thin, flexible sheet of material in a typical embodiment. The pad 12 includes hooks 14 as one portion of a hook and loop fastener (or optionally loops). Adhesive 18 is provided on the back surface 16, so that the pad 12 can attach to a reference surface 15. This reference surface could be surgical drape or skin of a patient, or some other underlying structure, which is sufficiently immobile to allow the system 10 to stabilize a catheter C when the grip assembly 20 attached to the catheter C is attached to the pad 12.

As an option, the pad 12 could be integrated with surgical drape, such as by providing a surgical drape with hooks 14 (or loops) on an upper surface thereof. In such a way, the surgical drape could directly function as the pad, 12 and interact with the grip assembly 20 attached to the catheter C (or directly to the catheter C, 320 in embodiments such as those disclosed in FIGS. 26-28). Surgical drape is typically large enough and/or dense enough that when it is draped over a patient and/or surfaces adjacent to a patient, the surgical drape tends to remain substantially immobile. Thus, when the pad 12 is attached to the surgical drape through the adhesive 18 on the back surface 16, the pad 12 also remains substantially immobile. Therefore, when the grip assembly 20 is attached to the catheter C, the catheter C remains immobile whenever the grip assembly 12 is brought into contact with the top surface 13 of the pad 12. Similarly, in embodiments which do not utilize the grip assembly 20, the catheter C can be brought adjacent to the pad 12 or other anchor for stabilization of the catheter C. Such an anchor can include some form of pad 12 or can merely be sufficiently large, and have appropriate shape (generally flat) that the anchor itself will resist rotation and/or translation of the catheter C relative to the anchor.

The grip assembly 20, in this example embodiment (FIGS. 1 and 2) has a central bore 22 passing along a central long axis of the grip assembly 20. The central bore 22 is large enough to allow the catheter C to pass through (along arrow B of FIG. 1). The grip assembly 20 includes an outer surface 24 with loops 26 (or hooks) thereon. The hooks 12 (or loops) and the loops 26 (or hooks) are formed to be complimentary to each other, so that when they are brought into contact with each other, they hold to each other. This holding force is sufficiently high that forces tending to cause the catheter C to translate or rotate are resisted, and the catheter C is stabilized and held in its substantially immobile and stabilized state. When the catheter C is referred to as being substantially immobile, it is meant that only small movements of the catheter C having no appreciable medically disadvantageous consequences are allowed to occur.

The holding force of the hook and loop fastener system between the hooks 14 and the loop 26 is sufficiently low that a person of average strength can easily grip the catheter C and/or grip assembly 20 and apply forces (opposite arrow A in FIGS. 1 and 2) to remove the grip assembly 20 and catheter C off of the pad 12. The grip assembly 20 can be repeatedly attached and detached to the pad 12 (along arrow A and opposite arrow A) as needed, such as during careful positioning of the catheter C during a medical procedure. Such movements of the catheter C during the medical procedure can include translation (along arrow T of FIG. 2) and rotation (along arrow R of FIG. 2). When the grip assembly 20 is in contact with the pad 12, such translation and rotation is resisted and the catheter C remains in its stabilized state and substantially immobile.

With particular reference to FIG. 3, one variation on the grip assembly 20 is disclosed which allows for lateral placement of the catheter C into the grip assembly 20 (along arrow D of FIG. 3), rather than requiring threading longitudinally as depicted in FIG. 1 (along arrow B). The grip assembly 20 includes a body 30 and 40 which threadably attached to each other. At least the body 30 is provided with a slit 38 extending between the first end 32 and a second end 34. A slit 38 also passes through male threads 36 at the second and 34.

The cap 40 includes female threads 46 which are formed to be complimentary with the threads 36 of the body 30, to allow the cap 40 to be threaded onto the second end 34 of the body 30. The cap 40 can optionally include a slit 48 and hinge 45 so that it also can be opened for lateral placement of the catheter C thereinto. However, typically the cap 40 would not have such a slit 48 and would be threaded longitudinally onto the catheter C. If the cap 40 is provided with such a slit 48, it would include some form of closure to keep it closed after the catheter C has been placed through the slit 48 into an interior of the cap 40. Such a closure could include a strap 47 attached to one side of the slit 48 and a snap 49 on the strap and a complementary snap 49 on an exterior of the cap 40, so that the strap 47 can be closed down over the cap 40 to keep the cap 40 from opening at the slit 48 after the catheter C has been placed therein.

Some form of catheter C engagement is associated with the grip assembly 20 which can tighten and hold the grip assembly 20 to the cap 40. In the variation of FIG. 3, a resilient liner 35 is provided surrounding the central bore 22 of the grip assembly 20. This resilient liner 35 has an inner diameter which is less than a diameter of the catheter C. Thus, a friction fit is provided between the body 30 and the catheter C when the halves of this variation of the body 30 are brought together to close the slit 38. The cap 40 is then threaded into the body 30 with the cap 40 acting to hold the two halves of the body 30 together.

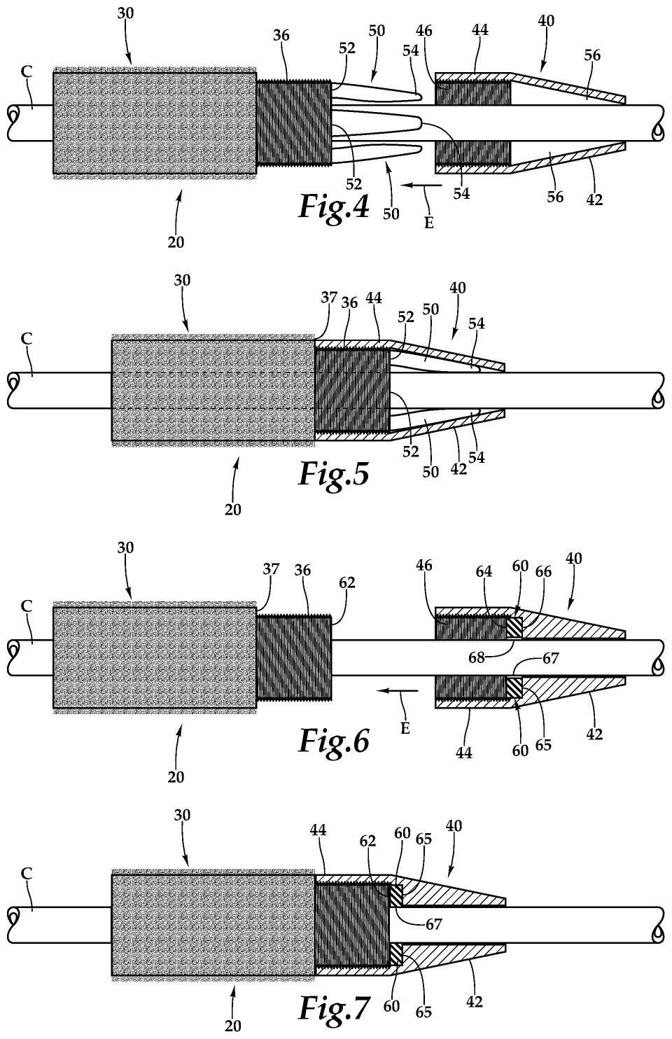

With a particular reference to FIGS. 4 and 5, a variation on the grip assembly 20 is disclosed featuring fingers 50 acting as a catheter engagement for the grip assembly 20. With this variation, the cap 40 preferably includes a tapering tip 42 at an end thereof opposite the body 30. A collar 44 is provided on the 40 adjacent to the body 30, which can include ribs on an outer surface thereof to facilitate gripping and rotating by hand. The collar 44 can thus thread the cat 40 and its associated female threads 46 onto the threads 36 of the body 30.

Fingers 50 extend from the second end 34 of the body 30 closest to the cap 40, typically adjacent to where the threads 36 terminate. These fingers 50 (such as four or more of them) extend from a root 52 at the second end 34 of the body 30 to tips 54 located opposite the roots 52. The tapering tip 42 of the cap 40 includes a hollow conical interior 56 in this variation, with a tapering inner surface. As the cap 40 is attached to the body 30 through rotation and engagement of the female threads 46 of the cap 40 with the threads 36 of the body 30, the fingers 50 are caused to slide along this tapering inner surface of the cap 40 within the taping tap 42 thereof, until the tips 54 of the fingers 50 are pressed firmly against the catheter C. The fingers 50 thus engage the catheter C when the cap 40 is tightened onto the body 30, and hold the grip assembly 20 firmly to the catheter C.

When the cap 40 is rotated in an opposite direction for removal from the body 30, the fingers 50 are released from this compression by the cap 40. The fingers 50 preferably have a natural state and which they are sufficiently resilient that they move away from the catheter C at least slightly when the cap 40 is at least partially removed from the body 30. At a minimum, force with which the fingers 50 engage the catheter C is reduced when the cap 40 is loosened, sufficient to allow the catheter C to be removed from the grip assembly 20.

By the rotation of the cap 40 onto enough of the body 30, the grip assembly 20 can be removably attached to the catheter C. Typically a surgical procedure would begin by first having the body 30 and cap 40 threaded onto the catheter C, by routing of an end of the catheter C through the central bore 22 of the grip assembly 20, through both the body 30 and the cap 40. The body 30 and cap 40 could remain loose on the catheter C initially, or could be attached (as described above) at a location along the length of the catheter C which is believed to be slightly outside of a transcutaneous access point where the catheter C goes from being outside of a body of the patient to inside the body of the patient. The cap 40 can be attached to the body 30 so the grip assembly 20 is attached to the catheter C at this point just outside of this transcutaneous access point. If such an estimation turns out to be incorrect, the cap 40 can be loosened from the body 30, and the grip assembly 20 merely slid along the catheter C somewhat in an appropriate direction to locate it where desired, and then the cap 40 is retightened to the body 30 for attachment of the grip assembly 20 to the catheter C where most appropriate.

The pad 12 is appropriately placed underneath the grip assembly 20 and attached either to skin of a patient or to surgical drape or to some other reference surface 15. The system 10 is then ready for use. When the surgical procedure is completed and stabilization is no longer required, the grip assembly 20 can be removed from the catheter C by merely reversing the procedure described above.

With particular reference to FIGS. 6 and 7, a further variation of the grip assembly 20 is described for catheter C engagement. In this variation, a compressible washer 60 is interposed between the body 30 and the cap 40. The fingers 50 are not provided in this variation. An interior of the tapering tip 42 in this variation is preferably filled and featuring a ledge 65 facing the second end 34 of the body 30, but spaced away from the second end 34 of the body 30. A compressible washer 60 is interposed between a face 62 at the second end 34 of the body 30 and this ledge 65, and generally within the cap 40 and adjacent to portions of the female threads 46 of the cap 40 which are closest to the tapering tip 42 of the cap 40. The cap 40 would not necessarily require that the tapering tap 42 have a conically tapering form, but instead could be a plane cylinder or some other shape, such as merely a longitudinal extension of the collar 44.

The compressible washer 60 has a first side 64 adjacent to the face 62 and a second side 66 adjacent to the ledge 65. This first side 64 and second side 66 of the compressible washer 60 are preferably parallel and spaced from each other by a thickness of the compressible washer 60. A hole 68 passes entirely through the compressible washer 60 from the first side 60 for the second side 66. Preferably this hole 68 has a diameter slightly greater than a diameter of the catheter C when the compressible washer 60 is in an original and natural state. When the compressible washer 60 has the catheter C threaded through the hole 68 thereof, a small gap 67 is provided between surfaces of the hole 68 and surfaces of the catheter C.

When the cap 40 is tightened down onto the body 30, the compressible washer 60 is caused to be compressed between the ledge 65 and the face 62. This causes the first side 64 and second side 66 of the compressible washer 60 to move toward each other. The compressible washer 60 is formed of materials which have an appropriate character such that the hole 68 is caused to be reduced in diameter as the compressible washer 60 is compressed between the ledge 65 and face 62. Such hole diameter reduction is greater than a size of the gap 67, so that surfaces of the hole 68 in a compressible washer 60 are caused to come into contact with and firmly grip the catheter C. The compressible washer 60 is also preferably formed from a material which has a sufficiently high coefficient of friction that when the compressible washer 60 is so compressed, friction forces cause the grip assembly 20 to remain attached to the catheter C. Such attachment can be readily reversed by merely unthreatening the cap 40 off of the body, 30, so that the compressible washer 60 can resiliently return to its original form, including the gap 67, and allowing the compressible washer 60 to be slid off of the catheter C along with the cap 40 and body 30.

With both of the variations of FIGS. 4 and 5, as well as the variation of FIGS. 6 and 7, tightening of the cap 40 upon the body 30 (along arrow E in FIGS. 4 and 6) causes attachment of the grip assembly 20 onto the catheter C. Similarly, removal of the cap 40 off of the body 30 is facilitated by movement in a direction opposite that of arrow E (FIGS. 4 and 6). The grip assembly 20 can thus be readily attached and detached onto the catheter C.

With particular reference to FIGS. 8-11, variations of the system 10 of this invention are described, which utilize the grip assembly 20 attached to the catheter C as part of the system 10, and feature a pad 12 which can optionally include hooks 14 as part of a hook and loop fastening system (with loops, 26 on the grip assembly 20) and can additionally (or in the alternative) include a mechanical fastener attached to the pad 12 for immobilization of the catheter C. If the hook and loop fastener is provided, then the mechanical fastener works with the hook and loop fastener for immobilization. If the hook and loop fastener is not provided, then the mechanical fastener attached to the pad 12 can act alone to immobilize the catheter C.

With the variation of FIG. 8 a mechanical fastener is provided in the form of a belt 80. The belt 80 includes a short strap 82 and a long strap 86 extending away from a central area where these straps 82, 86 are either joined together or at least are closest to each other and attached to the pad 12. A buckle 84 is provided on the short strap 82. The buckle 84 can connect to portions of the long strap 86 for selective attachment and detachment of the grip assembly 20 thereto. In one method of use, the grip assembly 20 can have loops 26 configured to engage with hooks 14 on the pad 12 for releasably gripping and stabilizing the catheter C during a medical procedure involving the catheter C. Once the medical procedure is over, the grip assembly 20 can be removed off of portions of the pad 12 spaced from the belt 80, and placed within a central portion of the belt 80. The hooks 14 of the pad 12 and the loops 26 of the grip assembly 20 would hold the grip assembly 20 to the pad 12. Furthermore, the belt 80 can be wrapped around the grip assembly 20 and the long strap 86 attached to the buckle 84 at or near an end of the short strap 82 and secured. The belt 80 thus can act in conjunction with the hooks 14 of the pad 12 and loops 26 of the grip assembly 20 to firmly hold the catheter C in this final post procedure position. The belt 80 can be readily removed when it is desired to move the catheter C again.

The belt 80 could optionally include hooks on an interior of the straps 82, 86, similar to those hooks 14 on the pad 12, to further allow for a secure connection to the loops 26 on the outer surface 24 of the grip assembly 20. As an option, the pad 12 could be free of hooks 14 and the grip assembly 20 could optionally be free of loops 26, and the belt 80 could be the only fastener holding the grip assembly 20 and the catheter C to the pad 12, through the belt 80.

With the variation of FIG. 9, the pad 12 is provided with a mechanical fastener in the form of a clamp 90. Clamp 90 includes a hinge 92 with a pair of arms 94 extending upwardly from the hinge 92 and facing each other. These arms 94 can, as an option, be at least semi-rigid and semi-cylindrical, so that they conform to significant portions of the catheter C when closed together. The arms 94 extended to tips 96. Fasteners 98 are typically provided, such as adjacent to the tips 96. One form of fasteners 98 would be magnets with a magnet of 98 oriented to come into contact with an opposite pole of the other magnet 98 for secure attachment of the tips 96 of the arms 94 of the clamp 90 to each other. As another alternative, one of the magnets could merely be a ferromagnetic material that is not a magnet, but is attracted to a magnet of the other fastener 98 on the other arm 94. Other fasteners 98 which could be used could including snaps, laces, suture receiving loops, or other fasteners. The clamp 90 would generally be utilized similar to the belt 80, either in conjunction with the hooks 14 of the pad 12 and the loops 26 of the grip assembly 20, or separate therefrom.

With the variation of FIG. 10, a clip 100 is provided on the pad 12. The clip 100 includes a base 102 attached to the pad 12, and with a pair of leaves 104 extending up from the base 102 on opposite sides of the base 102. The leaves 104 terminate through recurves 106 and tips 108. The tips 108 are spaced apart a greater distance than a diameter of the grip assembly 20, but the recurves 106 are provided closer to each other than a diameter of the grip assembly 20. A material from which the clip 100 is formed is elastic and sufficiently flexible that the grip assembly 20 can be pressed against the tips 108 and recurves 106 to snap into the clip 100 (along arrow A of FIG. 10). A reverse force can similarly remove the grip assembly 20 and catheter C from the clip 100.

In the variation of FIG. 11, a clasp 110 provides a mechanical fastener upon the pad 12, which can receive the grip assembly 20 therein for holding the catheter C to the pad 12. The clasp 110 has a side slot which allows for the grip assembly 20 to pass laterally into the clasp 110 (along arrow A of FIG. 11). The clasp 110 includes a base ring 112 and top ring 113, which are joined together by an outer side 114. The base ring 112 is mounted to the pad 12. The base ring 112 and top ring 113 are hollow at central portions of the clasp 110, allowing a button 115, inner wall 116 and foot 117 to reside inboard of the base ring 112 and top ring 113. A lateral opening into the clasp 110 is provided between an overhang 111 extending down from the top ring 113 and a lower lip 118 extending up from the foot 117, which foot 117 is formed along with the button 115 of a second piece inboard of the base ring 112 and top ring 113.

A spring 119 is located below the foot 117 and within the base ring 112. This spring 119 is preferably a compression spring, such as a helical compression spring or a resilient mass of material, to bias the button 115 and lower lip 118 and foot 117 toward an elevated position tending to make an entrance into the clasp 110 smaller than it is when the button 115 is depressed, causing the button 115 and foot 117 along with the lower lip 118 to move downwardly (along arrow F of FIG. 11). When the button 115 is released, the class 110 firmly grips the grip assembly 20. Otherwise, the clasp 110 is generally similar to the other mechanical fasteners optionally provided along with the pad 12, to further allow for engagement of the grip assembly 20 and catheter C, according to variations of the system 10 of this invention.

With particular reference to FIGS. 12-19, mechanical fasteners are disclosed, which are attachable to the pad 12 and which allow for securing of the catheter C without necessarily requiring the grip assembly 20. With these variations of the pad 12 and mechanical fastener, sizing of the mechanical fastener is reduced somewhat to accommodate the smaller size of the catheter C relative to the grip assembly 20 of previous variations. Also, the pad 12 would typically not include the hooks 14 thereon because the catheter C does not have loops thereon. However, the pad 12 could include hooks 14, such as if the catheter C is fitted with loops, such as in the embodiments disclosed and FIGS. 26-28 (described in detail below). Thus, the variations of FIGS. 12-19 could be utilized with or without hook and loop fasteners between the pad 12 and catheter C.

In FIG. 12 a sleeve 120 is affixed to the pad 12. This sleeve 120 includes a cylindrical recess 122 therein which is sized generally similar to but slightly smaller than a diameter of the catheter C. A base 124 of the sleeve 120 facilitates attachment to the pad 12. A slit 126 is provided which extends into the cylindrical recess 122. This slit 126 could be at the highest portion of the sleeve 120 or could be on a lateral side of the sleeve 120. The sleeve 120 is formed of an elastic material which is sufficiently flexible that the catheter C can push its way through the slit 126, with sides of the sleeve 120 on either side of the slit 126 flexing away from the slit 126 to allow the catheter C to snap thereinto. Because this cylindrical recess 122 is of a natural diameter slightly less than a diameter of the catheter C, the sleeve 120 securely holds catheter C therein. However, forces opposite arrow A of FIG. 12 can allow for removal of the catheter C from the sleeve 120. Thus, the catheter C can be transitioned between a stabilized state and a freely movable state.

In the variation of FIG. 13, and alternate clasp 130 is provided on the pad 12. This clasp 130 is similar to the clasp 110 of FIG. 11, except that it is sized to receive the catheter C rather than the grip assembly 20. As with the sleeve 120 of FIG. 12, the pad 12 could be provided with or without the hooks 14 thereon. A spring biases the alternate clasp 130 to grip the catheter C when it is passed laterally into the clasp 110 (along arrow A of FIG. 13). Then for removal, the button is depressed (along arrow F of FIG. 13) to allow for removal of the catheter C therefrom.

In the variation of FIG. 14, an alternate belt 140 is provided on the pad 12. This belt 140 is similar to the belt 80 of FIG. 8, except that it is sized to receive the catheter C rather than the grip assembly 20. The pad 12 could be provided with or without the hooks 14 thereon with this variation.

In the variation of FIG. 15, an alternate clamp 150 is provided on the pad 12. This clamp 150 is similar to the clamp 90 of FIG. 9, except that it is sized to receive the catheter C rather than the grip assembly 20. The pad 12 could be provided with or without the hooks 14 thereon with this variation.

In the variation of FIG. 16, an alternate clip 160 is provided on the pad 12. This clip 160 is similar to the clip 100 of FIG. 10, except that it is sized to receive the catheter C rather than the grip assembly 20. The pad 12 could be provided with or without the hooks 14 thereon with this variation.

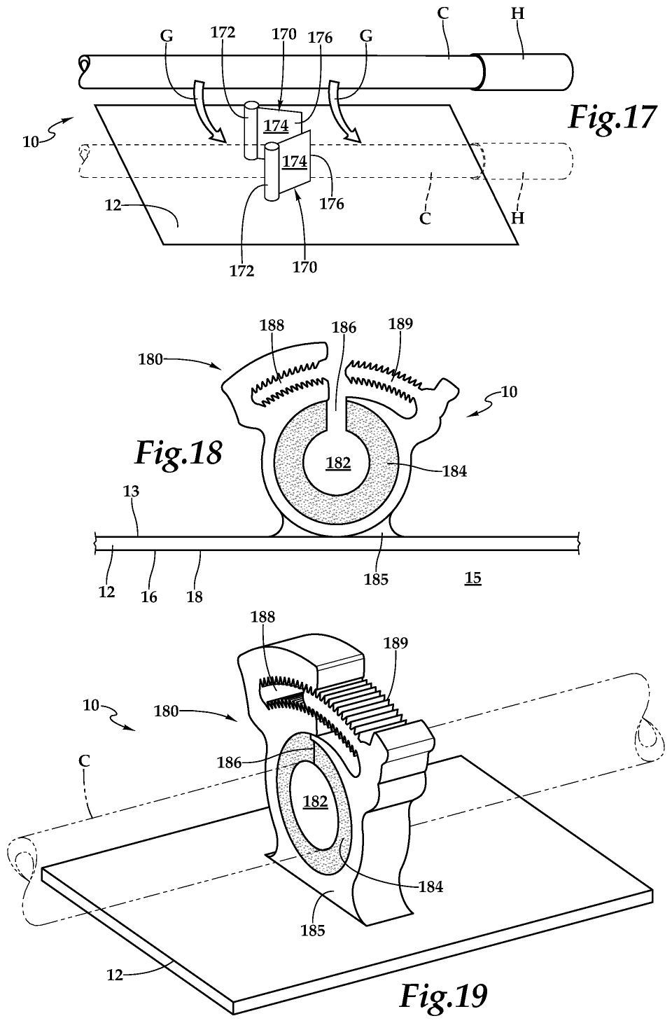

In the variation of FIG. 17, a mechanical fastener in the form of a gate 170 is provided on the pad 12. This gate 170 includes a pair of posts 172 extending up from the pad 12. Such extension is typically vertical and perpendicular to a top surface of the pad 12. Panels 174 extend laterally from the posts 172. These panels 174 are preferably biased toward a first position in which they are closer together than a diameter of the catheter C. However, the panels 174 can themselves flex outward or are pivotably attached to the post 172 so that the catheter C can be pushed through the panels 174 and between edges 176 of the panels 174 defining portions of the panels 174 closest to each other, so that the catheter C can be held between these edges 176 and within the gate 170. Such entering motion into the gate 170 would typically be in a downward and sweeping direction (along arrow G of FIG. 17). The catheter C can thus be held securely in position, but can be lifted out of the gate 170 when desired. The pad 12 could be provided with the hooks 14 or without the hooks 14 with this variation.

In the variation of FIGS. 18 and 19 a locking clip 180 is provided as a mechanical fastener attached to the pad 12. The locking clip 180 includes a bore 182 which can receive the catheter C therein by passage through a slit 186 extending into this bore 182. A base 185 of the locking clip 80 attaches the locking clip 180 to the top surface 13 of the pad 12. A back surface 16 opposite the top surface 13 can be fitted with an adhesive 18 to allow for attachment of the pad 12 to a reference service 15, such as surgical drape or skin.

The locking clip 180 is in many ways similar to the clamp 90 or clip 100 of FIGS. 9 and 10, or the alternate clamp 150 or alternate clip 160 of FIGS. 15 and 16. However, with the locking clip 180 resilient liner 184 is featured surrounding the bore 182. Also, an upper portion of the locking clip 180 is fitted with and arcuate toothed slot 188, which can receive an arcuate toothed finger 189 therein. Rotation of the arcuate toothed finger 189 into the arcuate slot 188 allows for tighter and tighter closure of the slit 186 and opposing sides of the slit 186 toward each other for securely holding the catheter C inboard of the resilient liner 184 of the bore 182.

For removal, the arcuate toothed finger 189 can be translated in a direction parallel with a longitudinal axis of the catheter C to be slid out of the arcuate toothed slot 188. Otherwise, the catheter C is securely held within the locking clip 180. The pad 12 can be provided with or without hooks 14 thereon. As with previous embodiments of FIGS. 12-17, catheter C could be directly fitted with loops 26, such as in the embodiments disclosed in FIGS. 26-28 (described in detail below).

With particular reference to FIGS. 20-25, embodiments are shown where the pad 12 is replaced with an anchor. Such an anchor facilitates catheter C stabilization because the anchor has sufficient mass and shape that it can merely be laid down on an underlying surface with the catheter C attached thereto, and resist catheter C translation and/or rotation. As an alternative, such an anchor can grip both the catheter C and surgical drape, or other adjacent structures, for further immobilization of the catheter C.

In one example embodiment of such an anchor, a cam clamp 200 is provided. The cam clamp 200 has a cam 202 which is biased away from a reference plate 206 and which cam 202 has teeth 204 on an end of the cam 202, which extend in a direction which allows the catheter C to translationally move laterally relative to the cam 202 and teeth, 204 in a first direction, generally parallel with the reference plate 206, but not move in a second direction opposite the first direction, and generally parallel with the reference plate 206. A torsion spring 208 built into or at least anchored to a post acts on the cam 202 to bias it toward the reference plate 206. Catheter C can be moved between the cam 202 and the reference plate 206 (along arrow A FIG. 20). The catheter C of this embodiment can move in a translating fashion in one direction, but not in the other direction. By orienting the cam clamp 200 either as shown or 180° rotated relative to the catheter C, the translational motion that is allowed (along arrow T of FIG. 21) can either be into or out of the body of a patient. The cam 202 rotates (along arrow I of FIG. 21) to lock and engage the catheter C for a mobilization of the catheter C when it moves in the direction opposite that of arrow T in FIG. 21.

The cam clamp 200 includes a handle. It has a generally planer form. It can thus really be rested on an underlying horizontal surface, and has sufficient mass that it will tend to hold the catheter C in a stabilized manner without substantial rotation, or translation. If desired, cam clamp 200 could be configured to attach to other structures, utilizing some form of fasteners such as adhesive, or one of various mechanical fasteners, such as those disclosed in detail above.

In FIGS. 22 and 23 a vee clamp 220 is disclosed as an anchor for selective gripping of the catheter C. The vee clamp 220 includes a fulcrum 222 opposite gripping loops, which are provided at ends of arms 224 extending away from the fulcrum 222. These arms 224 can rotate about the fulcrum 222, causing opposing semi-cylindrical troughs 226 to be brought toward each other (along arrow J of FIG. 23). These semi-cylindrical troughs 226 have radii of curvature matching radii of curvature of the catheter C (but typically slightly smaller, to facilitate griping of the catheter C therein). Toothed posts 228 can engage each other in releasable faction, similar to how a hemostat is used, to allow for holding of the vee clamp 220 in a closed orientation gripping the catheter C. As with the cam clamp 200, the vee clamp 220 would be sufficiently heavy that it can keep the catheter C substantially stabilized. Alternatively, vee clamp 220 could be attached to surgical drape or other structures to stabilize the catheter C most effectively. If desired, the semi-cylindrical troughs could be provided with a resilient liner to facilitate more firm gripping of the catheter C.

In FIGS. 24 and 25 a scissor clamp 240 is provided as an embodiment of anchor, according to an alternative embodiment of the system 10 of this invention. The scissor clamp 240 includes a pivot post 242 about which arms 244 and jaws 246 articulate. Motion of the handles 244 toward each other (along arrow J of FIG. 25) causes the jaws 246 associated with the arms 244 to be brought toward each other (along arrow K of FIG. 25). The jaws 246 are provided with troughs and optionally resilient liners and are appropriately sized to securely grip the catheter C. In particular, the jaws 246 (or at least resilient liners thereof), are preferably sized slightly smaller than a diameter of the catheter C, so that when the scissor clamp 240 is closed (FIG. 24) the catheter C is securely held between the jaws 246. Toothed posts 248 can engage each other to allow the scissor clamp 240 to temporally remain held together. These toothed posts 248 can be disengaged from each other, similar to with the vee clamp 220 of FIGS. 22 and 23 when opening of the scissor clamp 240 is desired.

With particular reference to FIGS. 26-28 an embodiment of the system 10 of this invention is disclosed where the pad 12 is provided with hooks 14, and loops are provided on the catheter C, 320 without requiring the entire grip assembly 20 (FIGS. 1 and 2). In particular, and in the variation disclosed in FIG. 26, a catheter 320 is provided which has an integrated grip surface 322 included therein. This grip surface 322 features loops similar to the loops 26 on the grip assembly 20, but affixed to an outer surface of the catheter 320. Such a customized catheter 320 would allow for simple and secure stabilization by merely pressing the catheter 320 down onto the pad 12.

With the variation of FIG. 27, the catheter C is unmodified, but a grip sheet 330 is provided. This grip sheet 330 is a flexible piece of paper, fabric or other thin flexible material, which has an inner surface 332 opposite an outer surface 334. The inner surface 332 would be provided with an adhesive to allow it to be attached to the catheter C. Outer surface 334 would include loops thereon, similar to the loops 26 on the grip assembly 20. When the grip sheet 330 has been attached to the catheter C, the catheter C can function similar to the catheter 320 of FIG. 26.

With the variation of FIG. 28, the catheter C is unmodified, but a grip clip 340 is provided which can snap onto the catheter C and fit the catheter C with a portion of a fastener similar to that provided with the grip sheet 330 or the catheter 320. In particular, the grip clip 340 has an inner surface 342 surrounding a central cylindrical recess, which is slightly smaller in size as the catheter C. A slit 343 extends longitudinally to provide access between an outer surface 344 of the grip clip 340 and the inner surface 342. The grip clip 340 is formed of a material which is sufficiently elastic, flexible and resilient that the catheter C can push its way through the slit 343 by expanding the slip 343, and then snap into the grip clip 340 adjacent to the inner surface 342. Typically, friction alone would hold the grip clip 340 to the catheter C. However, the inner surface 342 could be provided with an adhesive, or with a resilient liner to enhance friction forces, and hold the grip clip 340 to the catheter C. The outer surface 344 of the grip clip 340 would be provided with loops similar to the loops 26 associated with the grip assembly 20 (FIGS. 1 and 2). With the grip clip 340 attached to the catheter C, it will function similar to the catheter 320 of FIG. 26.

This disclosure is provided to reveal a preferred embodiment of the invention and a best mode for practicing the invention. Having thus described the invention in this way, it should be apparent that various different modifications can be made to the preferred embodiment without departing from the scope and spirit of this invention disclosure. When embodiments are referred to as “exemplary” or “preferred” this term is meant to indicate one example of the invention, and does not exclude other possible embodiments. When structures are identified as a means to perform a function, the identification is intended to include all structures which can perform the function specified. When structures of this invention are identified as being coupled together, such language should be interpreted broadly to include the structures being coupled directly together or coupled together through intervening structures. Such coupling could be permanent or temporary and either in a rigid fashion or in a fashion which allows pivoting, sliding or other relative motion while still providing some form of attachment, unless specifically restricted.

What is claimed is:

1: A system for securing a portion of a catheter, the system comprising in combination:
a grip, said grip configured to be attached to and hold to a portion of the catheter;
an anchor pad; and
a fastener interposed between said grip and said anchor pad for removable attachment of said grip and the catheter to said anchor pad, and holding of said catheter relative to said anchor pad.

2: The system of claim 1 wherein said grip includes a pathway through said grip for location of a portion of the catheter passing therethrough.

3: The system of claim 2 wherein said grip includes a slit extending laterally into said pathway, said slit allowing said catheter to pass into said pathway.

4: The system of claim 2 wherein said grip includes a first end with an entry into said pathway and a second and opposite said first end with an exit from said pathway, said entry and said exit sized to accommodate passage of the catheter into said pathway at said entry and out of said pathway at said exit.

5: The system of claim 2 wherein said grip includes a catheter engagement holding said grip firmly to said catheter.

6: The system of claim 5 wherein said catheter engagement removably attaches to the catheter through at least two parts of said grip which move relative to each other to releasably engage the catheter.

7: The system of claim 5 wherein said catheter engagement includes a compressible part which is compressed against the catheter to hold the catheter to said grip when said compressible part of said grip is compressed between two portions of said grip.

8: The system of claim 7 wherein said compressible part includes a plurality of fingers surrounding said pathway, said fingers flexible radially to engage with the catheter and hold the catheter when said fingers are flexed towards said catheter.

9: The system of claim 7 wherein said compressible part includes a compressible washer with a hole therein which hole is adapted to have the catheter pass therethrough before said compressible washer is compressed, and which compressible washer deforms and compresses against the catheter at said hole when forces are applied to said compressible washer to squeeze said compressible washer.

10: The system of claim 5 wherein said catheter engagement of said grip includes a first threaded part which is rotatable relative to a second threaded part, with rotation of said first threaded part relative to said second threaded part causing radial inward gripping forces to be applied by said grip upon the catheter located along said pathway of said grip.

11: The system of claim 1 wherein said fastener includes an adhesive.

12: The system of claim 1 wherein said fastener includes a mechanical fastener mounted upon said anchor pad, said clamp configured to releasably engage said grip.

13: The system of claim 12 wherein said mechanical fastener includes a belt sized to gird the catheter and the belt closable upon the catheter to hold the catheter to said anchor pad.

14: The system of claim 12 wherein said mechanical fastener includes a clasp which includes a spring which biases at least a first portion of said clasp toward a tight position, with a second reference portion of said clasp fixed to the anchor pad and with said first portion movable relative to said second portion to store energy in said spring and release said clasp from the catheter, and said first portion movable relative to said second portion to release energy in said spring and engage the catheter.

15: The system of claim 14 wherein said spring includes a compression spring.

16: The system of claim 14 wherein said first portion and said second portion engage the catheter through said grip.

17: The system of claim 12 wherein said mechanical fastener includes a clip which has a pair of resilient leaves spaced apart by a distance which receives and holds the catheter resiliently therein.

18: The system of claim 17 wherein said leaves are spaced apart by a distance which receives both the catheter and said grip therebetween.

19: The system of claim 12 wherein said fastener includes both said mechanical fastener and complementary hook and loop fasteners between said anchor pad and the catheter.

20: The system of claim 19 wherein the grip includes a complimentary one of a loop or hook fastener for attachment to a hook or loop fastener of the complimentary hook and loop fastener that is upon said anchor pad.
